(19) United States
(12) Patent Application Publication
(10) Pub. No.: US 2013/0071903 A1
(43) Pub. Date: Mar. 21, 2013

(54) METHOD FOR HEATING A FEEDSTOCK

(71) Applicant: Iogen Energy Corporation, Ottawa (CA)

(72) Inventors: Stephen A. Rowland, Brownsburgh-Chatam (CA); Torbjorn van der Meulen, Ottawa (CA)

(73) Assignee: IOGEN ENERGY CORPORATION, Ottawa (CA)

(21) Appl. No.: 13/623,207

(22) Filed: Sep. 20, 2012

Related U.S. Application Data
(60) Provisional application No. 61/536,805, filed on Sep. 20, 2011.

(51) Int. Cl. C13K 13/00; C12P 7/14; C09K 3/00; C12P 19/14

(52) U.S. Cl. 435/162; 435/99; 127/37; 252/183.11

(57) ABSTRACT

The present invention provides a method for producing a pretreated or hydrolyzed lignocellulosic feedstock. The method comprises feeding a lignocellulosic feedstock to a plug formation device and forming a feedstock plug therein. The plug or segments thereof are fed into an elongate chamber that comprises steam addition means for direct steam addition and a rotating shaft mounted co-axially within the chamber having one or more disintegrating elements mounted on it. Disintegrated feedstock particles are produced in the elongate chamber by the disintegrating elements. The disintegrated feedstock particles are heated by contact with the steam introduced through the steam addition means. The disintegrated feedstock particles are then treated in a reactor to produce the pretreated or hydrolyzed lignocellulosic feedstock. Further provided is a feedstock composition comprising disintegrated feedstock particles. Also provided are methods for reducing erosion on equipment by maintaining the discharge consistency from the plug formation device below 35 wt %.

### CROSS-REFERENCE TO RELATED APPLICATIONS

This application claims the priority benefit of provisional application No. 61/536,805, filed Sep. 20, 2011, which is incorporated herein by reference.

### FIELD OF THE INVENTION

The present invention provides an improved process for heating a feedstock prior to its entry into a downstream reactor. The present invention further provides an improved process for processing lignocellulosic feedstock while reducing erosion on process equipment.

### BACKGROUND OF THE INVENTION

There is increasing interest in producing fuel ethanol or other fermentation products from lignocellulosic feedstocks such as, for example, wheat straw, corn stover, and switch grass. An advantage of using these feedstocks is that they are widely available and can be obtained at low cost. In addition, lignocellulosic feedstocks are typically burned or landfilled, and so using them for ethanol production offers an attractive alternative to the costs of disposal. Yet another advantage of these feedstocks is that a byproduct of the conversion process, known as lignin, can be used as a fuel to power the process instead of fossil fuels. Several studies have concluded that when the entire production and consumption cycle is taken into account the use of ethanol produced from cellulose generates close to nil greenhouse gases.

One process for producing a fermentation product, such as ethanol, from lignocellulosic feedstocks is to carry out a pretreatment, followed by enzymatic hydrolysis of the cellulose to glucose. The pretreatment generally disrupts the fiber structure of the lignocellulosic feedstock and increases the surface area of the feedstock to make it accessible to cellulase enzymes. The pretreatment can be performed so that a high degree of hydrolysis of the xylan and only a small amount of conversion of cellulose to glucose occurs. The cellulose is hydrolyzed to glucose in a subsequent step that uses cellulase enzymes. Other pretreatment processes, such as certain alkali pretreatments, do not hydrolyze or result in limited xylan hydrolysis. Moreover, it is possible to hydrolyze both xylan and cellulose using more severe chemical treatment, such as concentrated acid hydrolysis.

Regardless of the method for producing fermentable sugar, the addition of water to the incoming feedstock to form a slurry is often carried out to facilitate the transportation and mechanical handling of the cellulosic feedstock. The slurry consists of lignocellulosic feedstock pieces or particles in water. Feedstock slurries can be most easily pumped when they have a consistency of about 1 and about 10 wt % undissolved dry solids.

However, for lignocellulosic conversion processes to be more economical, it would be desirable for them to operate at lower water content. The processing of feedstock of low water content has numerous advantages in various stages of the process, one of which is reductions in equipment size, which, in turn, reduces capital cost. Further benefits of low water content include reduced energy consumption including reductions in costs for pumping, heating, cooling and evaporating. Moreover, water usage costs can be reduced, which is especially advantageous in arid climates where water is at a premium.

A stage of the process that particularly benefits from low levels of water is pretreatment or other stages that require heat to treat the feedstock. During these treatments, the amount of energy required for heating up the feedstock slurry, upstream of the reactor, or within the reactor itself, is a direct function of the total mass of the feedstock slurry, including the water added for transportation of the feedstock. Operating a pretreatment or hydrolysis process with low levels of water can reduce the energy required for heating. Various methods are known for heating feedstock including indirect heating methods, such as heating jackets, the addition of heated water to a chamber such as disclosed in Canadian Patent Application No. 2,638,152, or the addition of steam to a reactor itself (U.S. Pat. No. 5,338,366).

One method for reducing water content, and the consequent energy requirements for heating, is to dewater the incoming feedstock slurry and form a compacted plug of feedstock prior to carrying out pretreatment or hydrolysis in a downstream reactor (see co-owned and co-pending WO 2010/022511, which is incorporated herein by reference). Plugs of feedstock can be produced by various devices, such as plug screw feeders and pressurized screw presses. Often the water content of the feedstock is reduced so that the solids content is high enough for plug formation to occur. Dewatering can take place within a plug formation device or dewatering and plug formation can be carried out in separate pieces of equipment. Alternatively, it is possible to eliminate dewatering upstream of plug formation if the feedstock solids content is already at a desired high consistency.

The plug that is formed can prove to be difficult to heat prior to its entry into the downstream reactor. Often the plug discharges into large segments, which can be 3-5 inches in diameter or even larger. Such large segments prevent rapid penetration of steam into the fibrous material and result in uneven temperature distributions. The inventors have recognized that uneven temperature distributions in the plug, or segments thereof, can result in overcooking or undercooking of the feedstock in the downstream reactor. Overcooking in the reactor can result in degradation of the feedstock, while undercooking can result in low xylose yield and difficult cellulose hydrolysis.

A further problem that arises during processes that utilize high consistency material is that the equipment is prone to erosion. Erosion damage to plug formation devices or other equipment exposed to high consistency feedstock slurry can be costly as it necessitates frequent repair or potentially even costly replacement of the equipment. The inventor has recognized that erosion damage on equipment could be particularly problematic with lignocellulosic feedstocks that contain relatively high levels of ash, such as cultivated crops, agricultural or sugar processing residues. Sugar cane straw and bagasse, which are currently of interest for second generation biofuel production, often contain quite significant amounts of ash. Although the ash can be removed by washing or leaching, such steps are often undesirable as they increase water usage in the process.

Thus, there is a need in the art for an improved process for heating a feedstock plug, or segments thereof, prior to entering a downstream reactor. There is also a need in the art for an improved process for reducing erosion on equipment when operating processes that involve forming a plug of material from non-woody lignocellulosic feedstocks.

### SUMMARY OF THE INVENTION

Disclosed herein are processes that overcome or ameliorate problems, or provide useful alternatives, in relation to known processes that form a plug of material from lignocellulosic feedstocks prior to pretreatment or hydrolysis.

According to certain embodiments of the invention, the present invention can overcome difficulties in heating a feedstock prior to its entry into a downstream reactor. In particular, by ensuring that a feedstock plug or segments thereof are disintegrated into particles in a heating chamber comprising disintegrating elements, a higher specific surface area can be achieved. As a consequence, more rapid penetration of steam into the fibrous material and more even temperature distributions may be achieved prior pretreatment or hydrolysis of the feedstock. By contacting the particles with steam in this manner, overcooking or undercooking of the feedstock in the downstream reactor can potentially be reduced, which, in turn, may improve the xylose yield and cellulose hydrolysis.

According to a first aspect of the invention, there is provided a method for producing a pretreated or hydrolyzed lignocellulosic feedstock comprising: feeding a lignocellulosic feedstock to a plug formation device and forming a feedstock plug therein; feeding the plug or segments thereof into an elongate chamber having at least a portion thereof that is cylindrical and which is preferably horizontally-oriented or essentially horizontally-oriented, the chamber having steam addition means for direct steam addition and a rotating shaft mounted therein having one or more disintegrating elements arranged thereon; producing disintegrated feedstock particles in the elongate chamber by the disintegrating elements; heating the disintegrated feedstock particles by contacting the particles with steam introduced through the steam addition means, wherein the operating pressure in the chamber is at least about 90 psia; and thereafter, pretreating or hydrolyzing the disintegrated feedstock particles in a reactor to produce the pretreated or hydrolyzed lignocellulosic feedstock.

According to a second aspect of the invention, there is provided a method, as set forth above, wherein the disintegrating elements are arranged on the shaft so as to sweep the inner surface of at least a region of the chamber. The disintegrating elements may continuously axially sweep the inner surface of at least a region of the chamber.

According to a third aspect of the invention, there is provided a method, as set forth above, wherein the disintegrating elements are pitched in the direction of feedstock movement through the heating chamber so as to facilitate conveyance of the feedstock through the heating chamber.

According to one embodiment of any of the foregoing aspects of the invention, the lignocellulosic feedstock is fed to a dewatering device, to produce a dewatered feedstock and the dewatered feedstock is then fed to the plug formation device. In a further embodiment of the invention, the feedstock is pressurized and then fed to the dewatering device and the pressure of the feedstock at the inlet of the dewatering device is greater than about 45 psia.

The disintegrating elements for disintegrating the feedstock may comprise a cut flight auger, a ribbon feeder, a sawtooth auger, blades, bars, paddles, pegs, arms, or a combination thereof. According to one embodiment of the invention, the disintegrating elements are located on the shaft in at least the mid-region of the chamber. The region of the shaft in the inlet section of the chamber may comprise a ribbon feeder, a cut flight auger or a sawtooth auger.

The disintegrating elements may project outwardly from the shaft and may be configured such that the outer edges of the disintegrating elements on the rotating shaft describe one or more circles that are concentric or essentially concentric in relation to the inner surface of the chamber.

According to a further embodiment of the invention, the speed of the outer edge of the disintegrating element that is closest to the inner surface of the chamber is about 200 m/min to about 1000 m/min. In a further embodiment of the invention, the speed of the outer edge of the disintegrating element that is closest to the inner surface of the chamber is about 450 m/min to about 800 m/min.

In a further embodiment of the invention, the distance between the inner surface of the chamber and the outer edge of the disintegrating element that is closest to the inner surface is less than 10 percent of the inside diameter of the chamber.

According to a further embodiment of the invention, the steam addition means comprises inlets for direct steam injection disposed along the length of the chamber. Preferably, the chamber does not contain an indirect heating jacket.

The pretreating or hydrolyzing may comprise the addition of chemical to the disintegrated feedstock particles. The chemical is typically acid or alkali.

The present invention also provides an improved process for reducing erosion on equipment when processing high consistency material from non-woody lignocellulosic feedstocks. As discussed, non-woody feedstocks often contain relatively high levels of ash compared to woody biomass and thus processes using these feedstocks are more prone to erosion damage on equipment, particularly equipment exposed to high consistency material, such as plug formation devices. The inventor has recognized that the impact of erosion damage on equipment when processing such feedstocks would be particularly pronounced when the consistency of the material is high. This is in contrast to woody materials, such as wood chips and pulp that contain relatively low levels of ash. Processes described in the literature that use wood chips or pulp as a feedstock for making ethanol can typically operate at higher consistency in the plug formation device.

Therefore, by operating at a lower consistency than that which is more prevalent in pulp and paper processes, erosion damage can be reduced, thereby resulting in savings in operating and capital costs. The consistency is controlled at the outlet of the plug formation device so that it remains below a threshold consistency value of 35 wt % undissolved dry solids, but above 20 wt % to maintain low water conditions.

Thus, according to another aspect of the invention, there is provided a method for producing a pretreated or hydrolyzed lignocellulosic feedstock comprising: (i) feeding a lignocellulosic feedstock in the form of a slurry to a plug formation device and forming a feedstock plug therein, wherein the plug or segments thereof exiting the plug formation device have an undissolved dry solids content between about 20 wt % and about 35 wt %; (ii) pretreating the lignocellulosic feedstock after step (i) to produce a pretreated lignocellulosic feedstock having an undissolved dry solids content of between about 15 wt % and about 30 wt %; (iii) enzymatically hydrolyzing the pretreated lignocellulosic feedstock to produce a solution comprising at least glucose; and (iv) fermenting at least the glucose to produce an alcohol, wherein the lignocellulosic feedstock is selected from cultivated crops, sugar processing residues and agricultural residues having an ash content of greater than 0.5% (w/w).

As demonstrated herein, the method set out above was effective in producing a cellulosic substrate from which high glucose yields can be recovered, while at the same time reducing erosion. In some embodiments of the invention, at least 70% of the cellulose in the pretreated lignocellulosic feedstock is converted to glucose. Preferably at least 80% or at least 90% of the cellulose in the pretreated lignocellulosic feedstock is converted to glucose.

The present invention also provides an improved method for producing a pretreated or hydrolyzed lignocellulosic feedstock that comprises a step of soaking the feedstock in an aqueous solution. The soaked feedstock may have an undissolved dry solids content of between about 1 wt % to about 12 wt %. Preferably, the soaking is carried out using an aqueous solution comprising an acid or alkali pretreatment chemical. A benefit of soaking the feedstock prior to pretreatment is that it can ensure uniform wetting of the biomass, which in turn helps achieve even cooking in the subsequent pretreatment or hydrolysis. The soaked feedstock is subsequently fed to a plug formation device to form a plug of material and the plug or segments thereof exiting the outlet of the plug formation device have an undissolved dry solids content that does not exceed 35 wt %, thereby reducing erosion on equipment.

Thus, according to a further aspect of the invention, there is provided a method for producing a pretreated or hydrolyzed lignocellulosic feedstock comprising: (i) soaking a lignocellulosic feedstock with an aqueous solution to produce a soaked lignocellulosic feedstock, wherein said lignocellulosic feedstock does not primarily contain wood chips or pulp; (ii) feeding the soaked lignocellulosic feedstock to a plug formation device and forming a feedstock plug therein, wherein the plug or segments thereof exiting the plug formation device have an undissolved dry solids content between about 20 wt % and about 35 wt %; (iii) disintegrating the plug or segments thereof to produce disintegrated feedstock particles and heating the disintegrated feedstock particles; and thereafter (iv) pretreating or hydrolyzing the disintegrated feedstock particles in a reactor to produce the pretreated or hydrolyzed lignocellulosic feedstock.

According to an embodiment of the invention, the soaked feedstock is partially dewatered in a dewatering device prior to being fed to the plug formation device. The partial dewatering may alternatively be carried out within the plug formation device itself.

Preferably, the lignocellulosic feedstock is sugar cane bagasse or sugar cane straw. Sugar cane straw and bagasse have been found to contain relatively high levels of ash. In one embodiment of the invention, the lignocellulosic feedstock has an ash content of between about 1.5% and about 15% (w/w). According to a further embodiment of the invention, the lignocellulosic feedstock is sugar cane bagasse or sugar cane straw having an ash content of between about 1.5% and about 15% (w/w), or between 1.5% and about 12% (w/w).

In some embodiments of the invention, at least 70% of the cellulose in the pretreated lignocellulosic feedstock is converted to glucose. Preferably at least 80% or at least 90% of the cellulose in the pretreated lignocellulosic feedstock is converted to glucose.

Without being limiting, by carrying out the foregoing methods that result in reduced erosion to process equipment, the use of a washing or leaching step may be reduced or even avoided altogether. This reduces water usage. However, it may be advantageous to remove a certain portion of the ash from the lignocellulosic feedstock to further reduce erosion or for other reasons. Thus, according to some embodiments of the invention, the lignocellulosic feedstock is not leached or washed prior to step (i) in order to remove greater than 50 wt % of the ash.

According to a further aspect of the invention, there is provided a lignocellulosic

feedstock composition comprising: (i) disintegrated lignocellulosic feedstock particles; (ii) about 15 to about 35 wt % undissolved solids, wherein the undissolved solids comprise between about 20 and about 60 wt % cellulose and between about 10 and about 30 wt % xylan; and (iii) a mineral or organic acid, wherein the feedstock particles are not primarily derived from wood chips or pulp, and wherein the pH of the feedstock composition is between about 0.5 and about 4.5. The temperature of the composition may be between about 100° C. and about 280° C.

According to a further embodiment of the invention, the lignocellulosic feedstock particles are derived from bagasse or sugar cane straw. According to yet a further embodiment of the invention, the lignocellulosic feedstock composition comprises about 15 wt % to about 30 wt % undissolved dry solids, or between about 20 wt % to about 30 wt % undissolved dry solids.

According to yet another aspect of the invention, there is provided a lignocellulosic feedstock composition comprising: (i) disintegrated lignocellulosic feedstock particles; (ii) about 15 to about 30 wt % undissolved dry solids, wherein the undissolved dry solids comprise between about 20 and about 60 wt % cellulose and between about 10 and about 30 wt % xylan; and (iii) a mineral acid, wherein the feedstock particles are not primarily derived from wood chips or pulp, and wherein the pH of the feedstock composition is between about 0.5 and about 3.5. The temperature of the composition may be between about 100° C. and about 280° C.

Further provided is a method comprising pretreating the foregoing lignocellulosic feedstock composition. The present invention also provides a pretreated lignocellulosic feedstock composition, wherein at least 70%, more preferably, 80% or 90% of the cellulose in the pretreated lignocellulosic feedstock, on a weight percent, can be converted to glucose, as measured when hydrolyzed with Trichoderma reesei cellulase enzymes, and wherein the pretreated lignocellulosic feedstock originates from sugar cane bagasse or sugar cane straw. The method for determining the digestability of the pretreated lignocellulosic feedstock with cellulase is set out in Example 4.

### BRIEF DESCRIPTION OF THE DRAWINGS

In the accompanying drawings,

FIG. 1 is a flow diagram of a method according to an embodiment of the invention;

FIG. 2 is a cross-section of a sawtooth auger utilized in a heating chamber according to an embodiment of the invention; and

FIG. 3 is graph showing the undissolved dry solids consistency (wt %) of a pretreated feedstock slurry produced in accordance with the method of invention measured over a one month time period of operation.

### DETAILED DESCRIPTION OF THE INVENTION

The following description is of a preferred embodiment by way of example only and without limitation to the combination of features necessary for carrying the invention into effect. The headings provided are not meant to be limiting of the various embodiments of the invention. Terms such as “comprises”, “comprising”, “comprise”, “includes”, “including” and “include” are not meant to be limiting. In addition, the use of the singular includes the plural, and “or” means “and/or” unless otherwise stated. Unless otherwise defined herein, all technical and scientific terms used herein have the same meaning as commonly understood by one of ordinary skill in the art.

### Feedstock and Feedstock Size Reduction

The feedstock for the method is a lignocellulosic material. By the term “lignocellulosic feedstock”, it is meant any type of plant biomass such as, but not limited to, plant biomass, including cultivated crops such as, but not limited to grasses, for example, but not limited to, C4 grasses, such as switch grass, cord grass, rye grass, miscanthus, reed canary grass, or a combination thereof, sugar processing residues, for example, but not limited to, bagasse, such as sugar cane bagasse, beet pulp, or a combination thereof, agricultural residues, for example, but not limited to, soybean stover, corn stover, rice straw, sugar cane straw, rice hulls, barley straw, corn cobs, wheat straw, canola straw, oat straw, oat hulls, corn fiber, or a combination thereof, forestry biomass for example, but not limited to, recycled wood pulp fiber, sawdust, hardwood, for example aspen wood, softwood, or a combination thereof. Furthermore, the lignocellulosic feedstock may comprise lignocellulosic waste material or forestry waste materials such as, but not limited to, newsprint, cardboard and the like. Lignocellulosic feedstock may comprise one species of fiber or, alternatively, lignocellulosic feedstock may comprise a mixture of fibers that originate from different lignocellulosic feedstocks. In addition, the lignocellulosic feedstock may comprise fresh lignocellulosic feedstock, partially dried lignocellulosic feedstock, fully dried lignocellulosic feedstock, or a combination thereof. Moreover, new lignocellulosic feedstock varieties may be produced from any of those listed above by plant breeding or by genetic engineering.

Preferably, the lignocellulosic feedstock is sugar cane bagasse or sugar cane straw. As would be appreciated by those of skill in the art, sugar cane straw includes the tops and leaves of sugar cane.

Lignocellulosic feedstocks comprise cellulose in an amount greater than about 20%, more preferably greater than about 30%, more preferably greater than about 40% (w/w). For example, the lignocellulosic material may comprise from about 20% to about 50% (w/w) cellulose, or any amount therebetween. Such feedstocks comprise hemicellulose, including xylan, arabinan, mannan and galactan. Furthermore, the lignocellulosic feedstock comprises lignin in an amount greater than about 10%, more typically in an amount greater than about 15% (w/w). The lignocellulosic feedstock may also comprise small amounts of sucrose, fructose and starch.

The lignocellulosic feedstock is typically subjected to size reduction by methods including, but not limited to, milling, grinding, agitation, shredding, compression/expansion, or other types of mechanical action. Size reduction by mechanical action can be performed by any type of equipment adapted for the purpose, for example, but not limited to size reduction devices selected from the group consisting of hammer mills, tub-grinders, roll presses, refiners and hydra-pulpers. Feedstock may be reduced to particles having a length of about 1/16 to about 8 inches, or any amount therebetween. The length of the reduced particles may also be such that at least about 90% by weight of the particles have a length less than about 5 inches or even shorter; for example, at least about 90% by weight of the particles may have a length less than about 4, about 3, about 2, about 1 or about ½ inches. Washing may be carried out to remove sand, grit and other foreign particles as they can cause damage to the downstream equipment. It will be understood that the lignocellulosic feedstock need not be subjected to size reduction, for example if the particle size of the feedstock is already between ½ to 8 inches.

For the purposes of this specification, the size of the feedstock particles is determined by image analysis using techniques known to those of ordinary skill in the art. An example of a suitable image analysis technique is disclosed in Igathinathane (Sieveless particle size distribution analysis of particulate materials through computer vision, Computers and Electronics in Agriculture, 2009, 66:147-158, the subject matter of which is hereby incorporated by reference), which reports particle size analyses of several different hammer milled feedstocks. The measurement may be a volume or a weight average length.

### Feedstock Consistency

Prior to feeding the lignocellulosic feedstock to the plug formation device, the amount of undissolved solids in the lignocellulosic feedstock may be adjusted to a desired consistency. The lignocellulosic feedstock can have an undissolved dry solids consistency of between about 1 wt % and about 40 wt % or between 4 wt % and about 20 wt %, upon entering the plug formation device and all ratios therebetween. The percent of undissolved dry lignocellulosic feedstock solids may be determined at the inlet of a plug formation device. The desired consistency is determined by factors such as pumpability, pipe-line requirements and other practical considerations.

The consistency (also referred to herein as undissolved dry solids or “UDS”) of the lignocellulosic feedstock is determined by filtering and washing a sample to remove dissolved solids and then drying the sample at a temperature and for a period of time that is sufficient to remove water from the sample of slurry or wet material, but does not result in thermal degradation of the feedstock solids. After the water removal, or drying, the dry solids are weighed and the weight of water in the sample of slurry or wet material is the difference between the weight of the sample of slurry or wet solids and the weight of the dry solids. The amount of undissolved dry solids (UDS) in an aqueous slurry is referred to as the consistency of the slurry. Consistency is expressed as the weight of dry solids in a weight of slurry, for example, as a ratio on a weight basis (wt:wt), or as a percent on a weight basis, for example, % (w/w), also denoted herein as wt %. The method for determining the consistency is set forth in Example 1.

Prior to feeding the lignocellulosic feedstock to a plug formation device, the feedstock may be soaked in an aqueous solution including water, or a solution comprising pretreatment chemical. A benefit of soaking the feedstock prior to pretreatment with a solution comprising pretreatment chemical is that it can ensure uniform impregnation of the biomass with the pretreatment chemical, which in turn helps achieve even cooking in the subsequent pretreatment. Uniform impregnation ensures that some material is not overcooked and degraded due to the high localized concentration of the pretreatment chemical, while other material is not undercooked, resulting in low xylose yield and difficult cellulose hydrolysis. Undercooking or overcooking of lignocellulosic feedstock can be particularly problematic when the pretreatment is conducted under medium or high solids consistency because the non-uniformity of the concentration of the pretreatment chemical and the temperature are more pronounced.

### Dewatering

The feedstock may be dewatered to increase the undissolved dry solids consistency within a desired range prior to plug formation. However, it should be understood that dewatering may not be required if the consistency of the feedstock is already at a desired level when it is fed to the plug formation device. The dewatering may involve removing water under pressure from the feedstock, or at atmospheric pressure, as discussed below.

A plug formation device may be configured to dewater the feedstock, although separate respective devices for dewatering and plug formation can be employed. Without being limiting, a plug formation device incorporating a dewatering section suitable for use in the invention may be a pressurized screw press or a plug screw feeder, as described in co-pending and co-owned WO 2010/022511, which is incorporated herein by reference. Water expressed from the lignocellulosic feedstock by the dewatering step may be reused in the process, such as for slurrying and/or soaking the incoming feedstock.

There are a variety of known devices that can be utilized to dewater the feedstock prior to plug formation. Examples include drainers, filtration devices, screens, screw presses, extruders or a combination thereof.

If the feedstock is subjected to dewatering under pressure, the pressure increase may be caused by one or more high pressure pumps. The pump or other feeding device increases the pressure of the feedstock prior to dewatering to e.g., about 45 psia to about 900 psia, or about 70 psia to about 800 psia or about 140 psia to about 800 psia. The pressure may be measured with a pressure sensor located at a feedstock inlet port on a dewatering device or a plug formation device that also dewaters the feedstock. Alternatively, the feedstock subjected to dewatering may be at atmospheric pressure or at a pressure below about 45 psia.

There may be an optional step of pre-draining the feedstock in order to drain out aqueous solution from the feedstock slurry at atmospheric pressure or higher. This pre-drained feedstock slurry can then be subjected to further dewatering.

### Plug Formation Devices

The plug formation can be considered an integration of lignocellulosic particles into a compacted mass referred to herein as a plug. Plug formation devices form a plug that acts as a seal between areas of different pressure. In embodiments of the invention, the plug seals against higher pressure in a device downstream of the plug. However, it should be understood that the pressure can be higher at the inlet of the plug formation device.

As mentioned previously, the plug formation device may dewater the feedstock, or this function may be carried out by an upstream dewatering device. Plug formation devices that dewater may comprise a housing or shell with openings through which water can pass. The plug formation device may be operated at atmospheric pressure or under pressure.

Without being limiting, the plug formation device may be a plug screw feeder, a pressurized screw press, a co-axial piston screw feeder or a modular screw device.

The plug of lignocellulosic feedstock may have a weight ratio of water to undissolved dry lignocellulosic feedstock solids of about 0.5:1 (67 wt % UDS) to about 5:1 (17 wt % UDS), or about 1:1 (50 wt % UDS) to about 4:1 (20 wt % UDS), or about 1.5:1 (40 wt % UDS) to about 4:1 (20 wt % UDS), or about 1.5:1 (40 wt % UDS) to about 3.5:1 (22 wt % UDS), and all ratios therebetween. The weight ratio of water to dry undissolved lignocellulosic feedstock solids or the weight % UDS in the plug of lignocellulosic feedstock or segments thereof may be determined by the method described in Example 1. Preferably, if the lignocellulosic feedstock is a non-woody feedstock, the undissolved dry solids content of the plug of lignocellulosic feedstock is below 35 wt %. As discussed, by operating below an undissolved dry solid content of 35 wt %, the process equipment is less prone to erosion due to ash present in such feedstocks. According to some embodiments of the invention, the undissolved dry solids content of the plug of lignocellulosic feedstock is between 20 wt % and 35 wt %, between 20 wt % and 32 wt %, between 22 wt % and 32 wt % or between 22 wt % and 30 wt %.

The non-woody feedstock may be a cultivated crop, a sugar processing residue or an agricultural residue. The non-woody feedstock will contain greater than 0.5 wt % ash (w/w), or more typically greater than 1 wt % ash (w/w). The ash includes, but is not limited to, silica, and salts of potassium, calcium and sodium. The salts may exist as carbonate, phosphate, chloride or other common salt forms. Magnesium and other minerals may be present as well depending on the source of the feedstock. In some embodiments of the invention, the ash content of the non-woody lignocellulosic feedstock is between about 0.5 wt % and about 18 wt %, between about 1 wt % and about 17 wt %, between about 1 wt % and about 15 wt % or between about 1 wt % and about 10 wt %. The ash content is measured as set forth in Example 2 and is determined relative to the oven dried weight of a feedstock sample.

### Disintegration and Steam Contact

After plug formation, the lignocellulosic feedstock is fed to a downstream elongate chamber, also referred to herein as a “high shear heating chamber” or a “heating chamber”, in which the feedstock is disintegrated into particles by disintegrating elements as it is conveyed therethrough. Typically, the heating chamber is horizontally-oriented or essentially horizontally-oriented. The disintegrated particles are heated by direct steam contact, which allows for efficient heat transfer.

At least a portion of the heating chamber is cylindrical. For example, at least a mid-region of the chamber may be cylindrical and the inlet and outlet regions of the chamber may be of a different shape, although chambers that are cylindrical along their entire axial length are preferred. It should be understood that the term “cylindrical” includes frusto-conical or other shapes that are substantially cylindrical.

The plug, or segments thereof, need not be fed directly into the heating chamber. Any of a variety of known devices may be positioned between the plug formation device and the heating chamber. Without being limiting, examples of such devices include mechanical restricting devices, restraining devices, scrapers and conveyors. It should be understood that the plug may break into segments as it is discharged from the plug formation device, or into other devices positioned downstream of the plug formation device, or as it is fed into the heating chamber.

The chamber comprises steam addition means for direct steam addition and a rotatable shaft mounted generally co-axially within the chamber comprising the one or more disintegrating elements that project outwardly from the shaft. Advantageously, it has been found that effective disintegration of a plug or plug segments can be achieved using disintegrating elements that impart energy into the plug or plug segments in a shearing action. As discussed below, operating parameters can be selected as required for optimal feedstock disintegration.

As used herein, the term “disintegrating elements” refers to members arranged on the shaft that convey the feedstock plug or segments thereof through the chamber and that impart sufficient shear to the feedstock, thereby producing disintegrated feedstock particles when the shaft rotates at a suitable speed. The disintegrating elements may comprise a cut flight auger, a ribbon feeder, a sawtooth auger, blades, bars, paddles, pegs, arms, or a combination thereof. It should be understood that the disintegrating elements can vary in length.

Disintegration involves transforming the plug or segments thereof into disintegrated particles. By disintegrated particles, it is meant that, in the heating chamber, clumps of fiber originating from the plug are broken down into their constituent particles, or that the clumps are substantially reduced in size in the high shear heating chamber. Without being limiting, if wheat straw is utilized, the clumps may be less than about 10 mm, or preferably less than about 5 mm in their least dimension.

The tip speed of the disintegrating elements is selected to cause feedstock disintegration and is generally higher than that utilized in mixing conveyors known in other industries. The tip speed of the disintegrating elements may be between about 200 m/min and about 1000 m/min, or between about 450 and about 800 m/min or any range therebetween. The shearing action is generally a function of the shape of the disintegrating elements, the number of disintegrating elements (if more than one disintegrating element is used) and tip speed. These parameters can be adjusted as required to achieve a desired rate of shear.

In some embodiments of the invention, the disintegrating elements are located on the shaft on at least a mid-region thereof. The inlet region of the shaft may comprise means for feeding and conveying the plug, or segments thereof, to the mid-region of the shaft where a more aggressive disintegration of the feedstock may occur. The outlet region of the shaft may comprise means for conveying the plug to the outlet of the chamber.

In further embodiments of the invention, the disintegrating elements are located on the inlet and/or outlet regions of the shaft. According to these embodiments, the elements on the inlet and/or outlet regions of the shaft not only convey the feedstock, but also disintegrate the feedstock. In some embodiments of the invention, the inlet region of the shaft comprises a ribbon feeder, a cut flight auger or a sawtooth auger. This configuration may improve the throughput capacity and minimize blockage upstream of the heating chamber.

Some or all of the disintegrating elements may be pitched in the direction of feedstock movement through the heating chamber so as to facilitate conveyance of the feedstock therethrough. That is, a disintegrating element may be mounted on the shaft at an angle off-set from a line drawn transverse to the heating chamber. Such a configuration may reduce the residence time distribution of the feedstock, which in turn minimizes overheating or underheating of the feedstock. For example, disintegrating elements may be mounted on the shaft at an angle that is off-set by between 0 and about 45° from a line drawn transverse to the shaft. For example, the disintegrating elements may be mounted on the shaft at an angle that is off-set by between 1 and about 45° from a line drawn transverse to the shaft, or at an angle that is off-set by between 5 and about 30° from a line drawn transverse to the shaft.

The steam addition means may comprise one or more inlets for direct steam injection. The introduction of steam along the length of the chamber at spaced-apart injection points allows for more even heating of the feedstock particles. The steam may be introduced through the feedstock inlet, inlets disposed along the length of the chamber, or a combination thereof. Additionally, chemical utilized for pretreatment or hydrolysis may be introduced into the heating chamber.

The operating pressure and temperature of the heating chamber will typically correspond to the pressure and temperature of the downstream reactor. The operating pressure of the chamber may be at least about 90 psia. Examples of suitable operating pressures include between about 90 and about 680 psia.

The temperature of the heating chamber will be greater than about 100° C. Examples of temperature ranges include between about 100° C. and about 280° C., or between about 160° C. and about 260° C.

In some embodiments of the invention, the disintegrating elements project outwardly from the shaft and are configured so that the outer edges thereof describe one or more circles that are concentric or essentially concentric in relation to the inner surface of the chamber. By the term “essentially concentric”, it is meant that the eccentricity of the one or more circles described by the outer edges is less than about 10% of the diameter of the heating chamber.

According to one embodiment of the invention, the distance between the inner surface of the chamber and the outer edge of the disintegrating element that is closest to the inner surface (also referred to herein as “clearance”) is less than about 10% of the inside diameter of the chamber. As mentioned previously, the lengths of the disintegrating elements can vary. Consequently, the clearance is measured at the outer edge of the disintegrating element that is closest to the inner surface of the chamber. In some embodiments of the invention, the clearance is between about 2% and about 8%, or between about 2.5% and about 6% of the inside diameter of the chamber.

The disintegrating elements are arranged on the shaft so as to sweep the inner surface of at least a region of the chamber. By sweeping the inner surface of the chamber in at least a region thereof, the disintegrating elements can reduce or remove scale build-up, including lignin deposits that can reduce the transport and mixing capacity of the heating chamber.

By the term “sweep”, it is meant that the distance between the inner surface of the chamber and the outer edge of the disintegrating element that is closest to the inner surface is less than 5% of the inside diameter of the chamber. By utilizing such a clearance, scale build-up can be removed from the inner surface of the chamber or the build-up can be reduced. Examples of suitable clearance ranges for sweeping include about 1.0% to about 5.0%, about 1.5% to about 4.5%, or about 2.0% to about 4.0%.

Furthermore, if discrete disintegrating elements are mounted on the shaft, e.g. blades, bars, paddles, pegs, arms, the spacing between adjacent elements, may be chosen so as to eliminate stagnant zones on the inner surface of the chamber between adjacent disintegrating elements where organic deposits accumulate on the inner surface of the chamber. For example, the disintegrating elements may overlap so as to provide continuous axial sweeping along at least a region of the chamber, thereby reducing or eliminating the stagnant zones.

The present invention also relates to a lignocellulosic feedstock composition comprising: (i) disintegrated lignocellulosic feedstock particles; (ii) about 15 to about 35 wt % undissolved dry solids, wherein the undissolved dry solids comprise between about 20 and about 60 wt % cellulose and between about 10 and about 30 wt % xylan; and (iii) a mineral or organic acid, wherein the feedstock particles are not primarily derived from wood chips or pulp, and wherein the pH of the feedstock composition is between about 0.5 and about 4.5.

By the phrase “does not primarily contain”, it is meant that the feedstock composition does not contain more than about 50 wt % feedstock particles from wood chips or pulp, preferably less than 40, 30, 20 or 10 wt %. In some embodiments of the invention, the feedstock composition does not primarily contain forestry biomass.

According to some embodiments of the invention, the undissolved dry solids content is 15, 16, 17, 18, 19, 20, 21, 22, 23, 24, 25, 26, 27, 28, 29, 30, 31, 32, 33, 34 or 35 wt %. The range of undissolved dry solids in the feedstock composition may include numerical limits of any of these values. According to further embodiments of the invention, the undissolved dry solids content is between about 20 and about 32 wt % or between about 18 and about 28 wt %.

According to further embodiments of the invention, the pH of the feedstock composition is 0.5, 1.0, 1.5, 2.0, 2.5, 3.0, 3.5, 4.0 or 4.5. The pH range of the feedstock composition may include numerical limits of any of these values. According to further embodiments of the invention, the pH is between about 0.5 and about 3.5 or between about 0.5 and about 3.0. The mineral acid may be sulfuric acid, sulfurous acid, hydrochloric acid, phosphoric acid or any combination thereof. Without being limiting, the acid may be sulfuric acid. The organic acid may be acetic acid.

The undissolved solids may contain 20, 25, 30, 35, 40, 45, 50, 55 or 60 wt % cellulose. The range of cellulose content in the undissolved solids may include numerical limits of any of these values. According to further embodiments of the invention, the cellulose content in the undissolved solids may be between about 30 and about 60 wt %.

The undissolved solids may contain 10, 15, 20, 25 or 30 wt % xylan. The range of xylan content in the undissolved solids may include numerical limits of any of these values. According to further embodiments of the invention, the xylan content in the undissolved solids may be between about 15 and about 30 wt %.

The temperature of the composition may be between about 100° C., 120, 140, 160, 180, 190, 200, 220, 240, 260 or 280° C. The temperature range may include numerical limits of any of these values. According to further embodiments of the invention, the temperature range is between 160 and 280° C.

### Pretreatment and Hydrolysis

After raising the temperature of the disintegrated feedstock particles in the heating chamber, they are pretreated or hydrolyzed.

The term “pretreatment” or “pretreat” means a process in which the lignocellulosic feedstock is reacted under conditions that disrupt the fiber structure and that increase the susceptibility or accessibility of cellulose within the cellulosic fibers for subsequent enzymatic or chemical conversion steps. A portion of the xylan in the lignocellulosic feedstock may be hydrolyzed to xylose and other hydrolysis products in a pretreatment process, although pretreatment processes that do not hydrolyze xylan are also encompassed by the invention. In embodiments of the invention, the amount of xylan hydrolyzed to xylose is more than about 50, about 60, about 70, about 80 or about 90 wt %.

By the term “pretreated feedstock”, it is meant a feedstock that has been subjected to pretreatment so that the cellulose contained in the cellulosic fibers has an increased susceptibility or accessibility to subsequent enzymatic or chemical conversion steps. The pretreated feedstock contains cellulose that was present in the feedstock prior to pretreatment. In some embodiments, at least a portion of the xylan contained in the lignocellulosic feedstock is hydrolyzed to produce at least xylose in a pretreatment.

The terms pretreatment or hydrolysis are not intended to be limited to the particular treatment methods disclosed herein. That is, they may or may not include the use of chemical (e.g., hydrothermal pretreatment) and the pretreatment or hydrolysis may be a multi-stage or a single stage process that produces fermentable sugar or prepares the feedstock for subsequent conversion to fermentable sugar. All or a portion of the polysaccharides contained in the feedstock may be converted to oligomeric or monomeric sugars, or a combination thereof during pretreatment or hydrolysis. If chemical is utilized during pretreatment or hydrolysis, it may include organic solvents, oxidizing agents, or inorganic acids or bases. Lignin may or may not be removed during the pretreatment or hydrolysis.

According to one embodiment of the invention, at least a portion of polysaccharides contained in the lignocellulosic feedstock is hydrolyzed to produce one or more monosaccharides.

Various types of reactors may be used to pretreat or hydrolyze the feedstock including two or more reactors, arranged in series or parallel.

According to one embodiment of the invention, the reactor is a vertical reactor, which may be either an upflow or a downflow vertical reactor. In another embodiment of the invention, the reactor is a horizontal or inclined reactor. The reactor may be equipped with an internal mechanism, such as a screw, conveyor, scraper or similar mechanism, for conveying the lignocellulosic feedstock therethrough and/or to aid in discharging the reactor.

The chemical for pretreating or hydrolyzing the feedstock may be added to the feedstock during a soaking process carried out prior to dewatering, prior to plug formation, into the heating chamber, into the plug formation device, into the reactor, or a combination thereof.

The pressure in the reactor is between about 90 psia and about 680 psia and any pressure therebetween. The pressure in the reactor may be measured with one or more pressure sensors. If the one or more reactors are configured so that there are different pressure levels within each, the pressure at the location where the feedstock enters the first reactor is considered herein to be the pressure of the reactor.

In some embodiments of the invention, the lignocellulosic feedstock is treated in the reactor under acidic conditions. For acidic conditions, a suitable pH is from about 0 to about 3.5 or about 0.2 to about 3 or about 0.5 to about 3 and all pH values therebetween.

The acids added to set acidic conditions in the reactor may be sulfuric acid, sulfurous acid, hydrochloric acid, phosphoric acid or any combination thereof. The addition of sulfurous acid includes the addition of sulfur dioxide, sulfur dioxide plus water or sulfurous acid. Organic acids may also be used, alone or in combination with a mineral acid.

The alkali added to set the alkaline conditions in the reaction zone may be ammonia, ammonium hydroxide, potassium hydroxide, sodium hydroxide or any combination thereof.

A suitable temperature and time of reaction in the reactor will depend upon a number of variables, including the pH in the reactor and the degree, if any, to which hydrolysis of the polysaccharides is desired.

Without being limiting, pretreatment of the lignocellulosic feedstock may take place under acidic or alkaline conditions. In an acidic pretreatment process, according to exemplary embodiments of the invention, the time in the pretreatment reactor may be from about 10 seconds to about 20 minutes or about 10 seconds to about 600 seconds or about 10 seconds to about 180 seconds and any time therebetween. The temperature may be about 150° C. to about 280° C. and any temperature therebetween. The pH for the pretreatment may be between about 0.5 and about 3, or between about 1.0 and about 2.0.

In an alkaline pretreatment process, the time in the reactor is from about 1 minute to about 120 minutes or about 2 minutes to about 60 minutes and all times therebetween, and at a suitable temperature of about 20° C. to about 220° C. or about 120° C. to about 220° C. and all temperatures therebetween.

Ammonia fiber expansion (AFEX), which is an alkali pretreatment method, may produce little or no monosaccharides. Accordingly, if an AFEX treatment is employed in the reaction zone, the hydrolyzate produced from the reaction zone may not yield any monosaccharides.

According to the AFEX process, the cellulosic biomass is contacted with ammonia or ammonium hydroxide, which is typically concentrated, in a pressure vessel. The contact is maintained for a sufficient time to enable the ammonia or ammonium hydroxide to swell (i.e., decrystallize) the cellulose fibers. The pressure is then rapidly reduced which allows the ammonia to flash or boil and explode the cellulose fiber structure. The flashed ammonia may then be recovered according to known processes. The AFEX process may be run at about 20° C. to about 150° C. or at about 20° C. to about 100° C. and all temperatures therebetween. The duration of this pretreatment may be about 1 minute to about 20 minutes, or any time therebetween.

Dilute ammonia pretreatment utilizes more dilute solutions of ammonia or ammonium hydroxide than AFEX. Such a pretreatment process may or may not produce any monosaccharides. Dilute ammonia pretreatment may be conducted at a temperature of about 100 to about 150° C. or any temperature therebetween. The duration for such a pretreatment may be about 1 minute to about 20 minutes, or any time therebetween.

When sodium hydroxide or potassium hydroxide are used in the pretreatment, the temperature may be about 100° C. to about 140° C., or any temperature therebetween, the duration of the pretreatment may be about 15 minutes to about 120 minutes, or any time therebetween, and the pH may be about pH 11 to about 13, or any pH value therebetween.

Alternatively, an acidic or alkaline hydrolysis process may be operated under conditions sufficiently harsh to hydrolyze cellulose to glucose and other products.

Acidic hydrolysis that is harsh enough to hydrolyze xylan and cellulose may be conducted for about 10 seconds to about 20 minutes, or any time therebetween. The temperature may be between about 180° C. and about 260° C., or any temperature therebetween. The pH may be between 0 and about 1 or any pH therebetween.

Alkali hydrolysis that is harsh enough to hydrolyze xylan and cellulose may be conducted at about 125° C. to about 260° C., or about 135° C. to about 260° C., or about 125° C. to about 180° C., or any temperature therebetween, for about 30 minutes to about 120 minutes, or any time therebetween and at about pH 13 to about 14, or any pH therebetween.

The pretreated or hydrolyzed feedstock may be discharged into a discharge device such as a screw discharger, a swept orifice discharger, a rotary discharger, a piston type discharger and the like. Two or more reactors, arranged in series or in parallel, may be used.

The hydrolyzed or pretreated feedstock exiting the reaction zone may be depressurized and flash cooled, for example to between about 30° C. and about 100° C. In one embodiment of the invention, the pressure is reduced to about atmospheric. The cooling and depressurization may be carried out by one or more flash vessels.

The undissolved dry solids of the pretreated feedstock slurry may be between about 15 and about 30 wt % or between about 15 and about 25 wt %.

### Enzymatic Hydrolysis and Fermentation

If the hydrolyzed or pretreated feedstock exiting the reactor contains cellulose, it may be subjected to cellulose hydrolysis with cellulase enzymes. By the term “cellulase enzymes”, “cellulase”, or “enzymes”, it is meant enzymes that catalyze the hydrolysis of cellulose to products such as glucose, cellobiose, and other cello-oligosaccharides. Cellulase is a generic term denoting a multienzyme mixture comprising exo-cellobiohydrolases (CBH), endoglucanases (EG) and β-glucosidases (βG) that can be produced by a number of plants and microorganisms. The process of the present invention can be carried out with any type of cellulase enzymes, regardless of their source.

Optionally, prior to the enzymatic hydrolysis, the sugars arising from pretreatment are separated from the unhydrolyzed feedstock components in the pretreated feedstock slurry. Expedients for carrying out the separation include, but are not limited to, filtration, centrifugation, washing or other known processes for removing fiber solids or suspended solids. The aqueous sugar stream may then be concentrated, for example, by evaporation, with membranes, or the like. Any trace solids are typically removed by microfiltration.

In one embodiment, the aqueous sugar stream separated from the fiber solids is fermented to produce a sugar alcohol by a yeast or bacterium. The sugar alcohol may be selected from xylitol, arbitol, erythritol, mannitol and galactitol. Preferably, the sugar alcohol is xylitol. Alternatively, the sugar is converted to an alcohol, such as ethanol or butanol, by fermentation with a naturally-occurring or recombinant bacterium or fungus. It should be understood that the invention is not limited to the particular chemical that can be produced from fermentable sugar or the particular method employed for producing same.

Generally, a temperature in the range of about 45° C. to about 55° C., or any temperature therebetween, is suitable for most cellulase enzymes, although the temperature may be higher for thermophilic cellulase enzymes. The cellulase enzyme dosage is chosen to achieve a sufficiently high level of cellulose conversion. For example, an appropriate cellulase dosage can be about 5.0 to about 100.0 Filter Paper Units (FPU or IU) per gram of cellulose, or any amount therebetween. The FPU is a standard measurement familiar to those skilled in the art and is defined and measured according to Ghose (1987, Pure and Appl. Chem. 59:257-268). The dosage level of β-glucosidase may be about 5 to about 400 β-glucosidase units per gram of cellulose, or any amount therebetween, or from about 35 to about 100 β-glucosidase units per gram of cellulose, or any amount therebetween. The β-glucosidase unit is also measured according to the method of Ghose (supra).

The enzymatic hydrolysis of the cellulose continues for about 24 hours to about 250 hours, or any amount of time therebetween, depending on the degree of conversion desired. The slurry thus produced is an aqueous solution comprising glucose, xylose, other sugars, lignin and other unconverted, suspended solids. Other sugars that may be produced in the reaction zone may also be present in the aqueous solution. The sugars are readily separated from the suspended solids and may be further processed as required, for example, but not limited to, fermentation to produce fermentation products, including, but not limited to ethanol or butanol by yeast or bacterium. If ethanol is produced, the fermentation may be carried out with a yeast, including, but not limited to Saccharomyces cerevisiae.

The dissolved sugars that are subjected to the fermentation may include not only the glucose released during cellulose hydrolysis, but also sugars arising from a pretreatment, namely xylose, glucose, arabinose, mannose, galactose or a combination thereof. These sugars may be fermented together with the glucose produced by cellulose hydrolysis or they may be fed to a separate fermentation. In one embodiment of the invention, such sugars are converted to ethanol, along with the glucose from the cellulose hydrolysis, by a Saccharomyces cerevisiae yeast strain having the capability of converting both glucose and xylose to ethanol. The Saccharomyces cerevisiae strain may be genetically modified so that it is capable of producing this valuable byproduct (see, for example, U.S. Pat. No. 5,789,210, which is incorporated herein by reference), although it has been reported that some Saccharomyces cerevisiae yeast strains are naturally capable of converting xylose to ethanol.

### EXAMPLES

### Example 1

### Determination of the Undissolved Solids Concentration in a Lignocellulosic Feedstock Slurry

The determination of the undissolved dry solids (UDS) content in a slurry is carried out as follows.

A fixed amount of slurry is dispensed into a plastic weigh dish and the slurry weight is recorded accurately using an analytical scale. A 1.6 μm filter paper circle, appropriately sized for a Buchner funnel, is placed in an aluminum weighing tin and the combined weight of the tin and filter paper is recorded. After transferring the pre-weighed filter paper to the Buchner funnel, the pre-weighed slurry is passed through the filter paper to isolate the solids. Small volumes of de-ionized water are used to ensure that the solids are quantitatively transferred from the weigh dish to the Buchner funnel. The solids are then washed using excess deionized water, after which the washed sample and filter paper are transferred into the pre-weighed aluminum tin. Care should be taken to ensure the solids are quantitatively transferred. After drying the aluminum tin in a 105° C. oven overnight, the contents are weighed accurately and the UDS is quantified by determining, as a percent or ratio, the number of grams of dry solids per gram of slurry.

### Example 2

### Determination of the Ash Content of a Lignocellulosic Feedstock

The amount of ash is expressed as the percentage of residue remaining after dry oxidation at 575° C. in accordance with NREL Technical Report NREL/TP-510-42622, January 2008, which is incorporated herein by reference. The results are reported relative to a 105° C. oven dried sample (dried overnight).

In order to determine ash the content, a crucible is first heated without any sample in a muffle furnace for 4 hours at 575±25° C., cooled and then weighed. After heating, the crucible is cooled and then dried to constant weight, which is defined as less than a ±3 mg change in the weight of the crucible upon one hour of re-heating the crucible at 575±25° C.

The sample analyzed is a 105° C. oven dried specimen. The weight of the oven dried sample is recorded after drying at 105° C. overnight in an oven and this weight is referred to as “oven dried weight” or “ODW”. The dried, weighed sample is placed in the crucible and ashed to constant weight in a muffle furnace set to 575±25° C. The crucible and ash are weighed subsequent to ashing and the percentage ash is determined on an ODW basis. The ash is quantified by determining, as a percent, the number of grams of ash per gram of oven dried sample.

### Example 3

### Feedstock Dewatering, Plug Formation, Plug Disintegration and Pretreatment System

The following describes a system for producing a pretreated feedstock in accordance with embodiments of the invention.

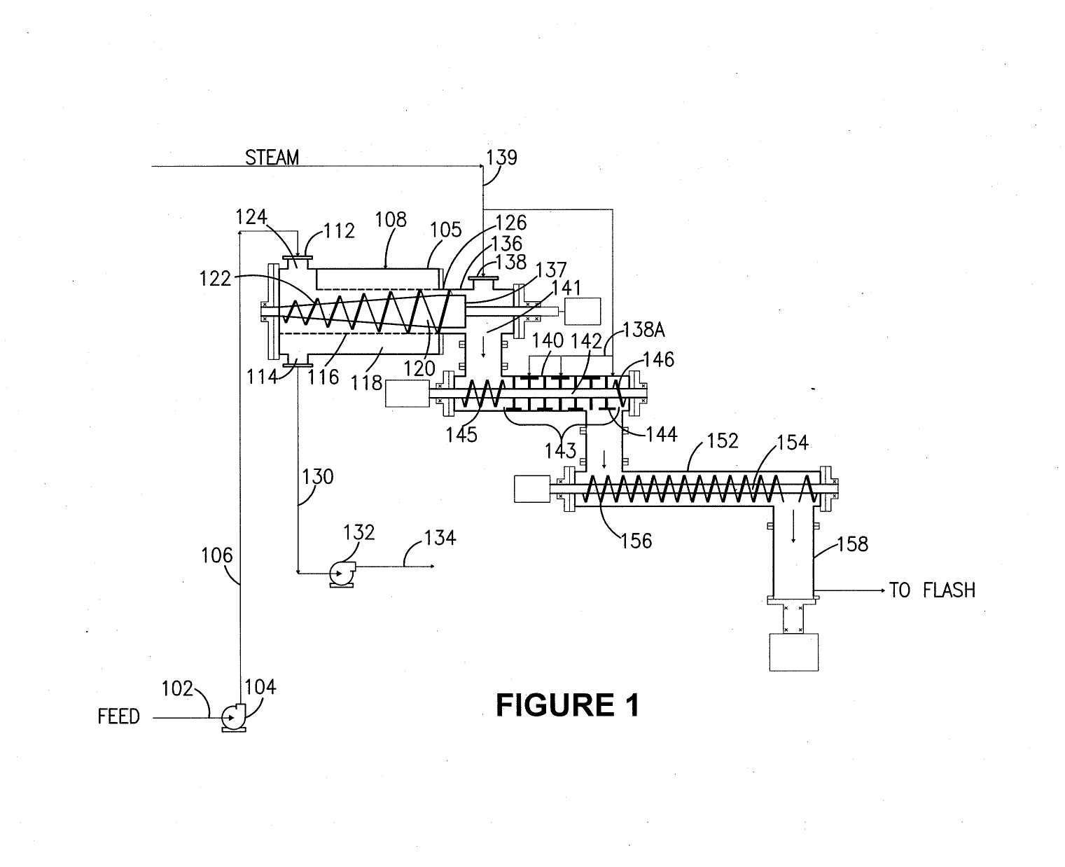

With reference to FIG. 1, a slurry of lignocellulosic feedstock having a consistency of about 1% to about 10% (w/w), preferably about 3% to about 5% (w/w) in slurry line 102 is pumped by means of pump 104 through in-feed line 106 into pressurized dewatering screw press indicated by general reference number 108. Pressurized dewatering screw press 108 comprises a solid shell 105 having a feedstock inlet port 112 and a pressate port 114. In-feed line 106 feeds lignocellulosic feedstock into the dewatering screw press 108 through the feedstock inlet port 112 at a pressure of, e.g., about 70 psia to about 900 psia. The pressure may be determined by measuring the pressure with a pressure sensor located at feedstock inlet port 112.

A screen 116 is disposed within shell 105 to provide an outer space 118 between the screen and the inner circumference of shell 105. A screw 120 is concentrically and rotatably mounted within the screen 116. The flights 122 of the screw 120 are of generally constant outside diameter and attached to a screw shaft with a core diameter that increases from the inlet end 124 to the outlet end 126 of the pressurized dewatering screw press 108.

Water and any other liquids, including dissolved solids, which have been expressed from the lignocellulosic feedstock slurry are withdrawn into the space 118, which serves as a collection chamber for the withdrawn water. The space 118 is connected through the pressate port 114 to a turbine 132 that draws withdrawn water through a pressate line 130. The withdrawn water, or pressate, may then be sent to a pressate return slurry make-up system (not shown) via line 134.

The partially dewatered lignocellulosic feedstock exits the dewatering and plug formation zone of the screw press 108 at the outlet end 126. The ratio of the weight of water to dry lignocellulosic feedstock solids in the partially dewatered lignocellulosic feedstock may be in the range of about 1.5:1 (67 wt % UDS) to about 4:1 (20 wt % UDS) exiting the dewatering and plug formation zone. The weight ratio of water to dry lignocellulosic feedstock solids in the dewatered lignocellulosic feedstock or the percent undissolved dry solids is determined by collecting a sample of the feedstock from, e.g., outlet end 126 of the screw press, and determining the weight ratio or weight % UDS in the sample by the method described in Example 1 above. Most preferably, the consistency of the feedstock plug or segments thereof at the outlet do not exceed 35 wt % UDS in order to reduce erosion on the screw press 108.

The outlet end 126 of the pressurized screw press 108 is operatively connected to a plug zone 136. A plug of the partially dewatered lignocellulosic feedstock is forced through the plug zone 136 and is discharged at plug outlet 137. There may also be a restraining device (not shown) at the plug outlet 137.

A steam inlet port 138 and/or ports 138A are supplied by a source of steam via steam inlet line 139. The plug of partially dewatered feedstock, which contains water in the range of about 0.5 to about 5 times the weight of the dry feedstock solids, is fed into a high shear heating chamber 140 via a feed chamber 141.

In the high shear heating chamber 140, the feedstock plug, or segments thereof, are disintegrated into particles, which are heated by direct steam contact via steam introduced through line 139 and/or ports 138A. Steam may also be introduced into the body of the heating chamber 140. As mentioned previously, the plug may break into segments as it is discharged from the pressurized screw press 108, or as it is fed into other devices positioned downstream of the screw press 108.

The heating chamber 140 is a cylindrical, horizontally-oriented device having a concentric, rotatable shaft 142 mounted co-axially in the chamber. The concentric shaft 142 comprises a plurality of disintegrating elements 143 mounted on its mid-region and that project radially therefrom. Some disintegrating elements comprise a distal end 144 that is “T-shaped” for sweeping the inner surface of the chamber 140, as described below. The inlet region of the shaft 142 comprises an inlet auger 145 for conveying the plug, or segments thereof, into the mid-region of the chamber. In addition, an outlet auger 146, with opposite pitch, is provided in an outlet region of the shaft 142 for discharging heated, disintegrated feedstock produced in the heating chamber 140 into a pretreatment reactor 152.

Shearing action is imparted to the feedstock plug or segments thereof, in the heating chamber 140 by the plurality of disintegrating elements 143. The tip speed of the shaft is such that the feedstock segments are disintegrated and is typically within a range of between 450 m/min to about 800 m/min so as to achieve optimal disintegration. The extent of shearing action is largely a function of the number and shape of the disintegrating elements times the tip speed. During disintegration, the feedstock plug or segments thereof are broken down into small particles.

Each disintegrating element is configured so that the clearance between the inner surface of the chamber 140 and the outer edge of the distal “T-shaped” end 144 of each disintegrating element is less than 4 percent of the inside diameter of the chamber 140. Such a clearance allows the disintegrating elements 143 to sweep the inner surface of the chamber 140.

Moreover, the disintegrating elements 143 are arranged on the shaft 142 so that there is continuous axial sweeping of the inner surface of the chamber 140. According to this embodiment of the invention, the end portions of each “T-shaped” disintegrating element overlap corresponding end portions of an adjacent T-shaped element. This allows the area swept by each T-shaped element to overlap the area swept by an adjacent T-shaped element so that there are no stagnant zones for organic deposits to accumulate on the inner surface of the chamber.

According to another embodiment of the invention, the disintegrating elements are “Y-shaped”. In addition, a combination of “Y-shaped” and “T-shaped” disintegrating elements may be arranged on the shaft.

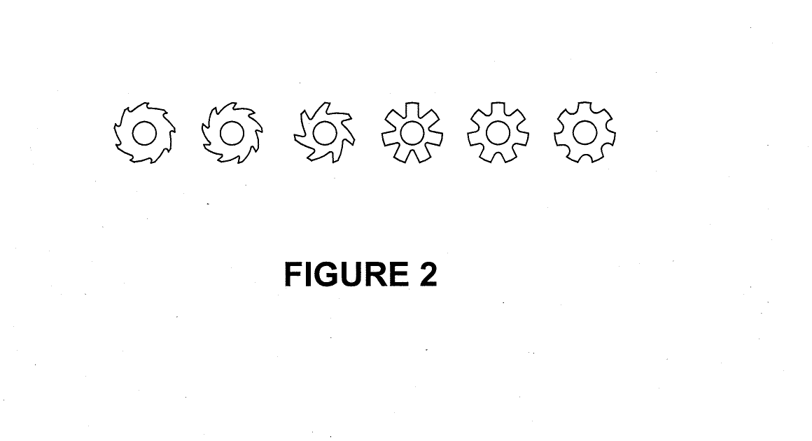

The auger 145 for conveying the plug, or segments thereof, into the mid-region of the chamber 140 may be sawtooth auger. Cross-sections of various auger configurations suitable for use in the invention are shown in FIG. 2. The provision of such an auger at the inlet region facilitates conveyance of the plug, or segments thereof, through the heating chamber 140. In addition, a sawtooth auger functions to disintegrate the feedstock plug or segments as it enters the heating chamber.

The heated, disintegrated feedstock is discharged from the heating chamber 140 into the pretreatment reactor 152, which comprises a cylindrical, horizontally-oriented vessel within which is mounted a screw conveyor 154 having flights 156. The pretreatment reactor 152 operates at a pressure of about 90 psia to about 680 psia, a pH of about 0.5 to about 3.0 and a temperature of about 160° C. to about 260° C. The lignocellulosic feedstock is treated in the reactor for a time of about 10 to about 600 seconds. The desired pH in the reactor 152 may be obtained by adding acid to the lignocellulosic feedstock prior to the inlet of the pressurized screw press.

A discharge device 158 discharges the pretreated feedstock from the pretreatment reactor 152. Subsequently, the pretreated feedstock is flashed in a flash vessel or vessels (not shown) to cool it before enzymatic hydrolysis.

### Example 4

### Production of a Pretreated Feedstock with Enhanced Enzymatic Digestibility to Cellulase, while Reducing Equipment Erosion

The method described in this example involves soaking a lignocellulosic feedstock in an acidic aqueous solution at low consistency and subsequently dewatering the soaked feedstock slurry using a pressurized screw press to an undissolved solids consistency of 28 wt %. The plug segments exiting the screw press were disintegrated in a heating chamber and subsequently pretreated at elevated temperature and pressure.

By keeping the UDS no higher than 28 wt % at the plug zone of the screw press, excessive wear and tear on the screw press is avoided. The highest UDS consistency occurs in the plug zone of the pressurized screw press and thus it is at this stage that the consistency is controlled to reduce erosion. The subsequent pretreatment results in a feedstock slurry of 20 wt % UDS. The results below show that the pretreatment was effective in producing a pretreated feedstock slurry from which a high glucose yield can be recovered under low water conditions.

Wheat straw was subjected to particle size reduction and soaked in an acidic solution at a pH of 1.4. Wheat straw has been reported to contain an ash content of 3.1% silica and 4.9% non-silica salts. (See co-owned U.S. Pat. No. 7,754,457).

With reference to FIG. 1, the soaked feedstock slurry was pumped by means of pump 104 through in-feed line 106 into the pressurized dewatering screw press indicated by general reference number 108. The pressurized dewatering screw press 108 is operated so that the plug segments exiting the device have an UDS of 28 wt %. As discussed, by operating at this dry solids consistency, erosion on the screw press due to the ash content of the feedstock can be reduced.

The plug segments are fed into a high shear heating chamber 140 via a feed chamber 141. In the high shear heating chamber 140, the feedstock segments exiting the device, are disintegrated into particles. The feedstock particles are heated by direct steam contact via steam introduced through line 139 and/or ports 138A.

The heated, disintegrated feedstock is discharged from the heating chamber 140 into a pretreatment reactor. The pretreatment is conducted at the pH, temperature and time set forth in co-owned U.S. Pat. No. 7,754,457, which is incorporated herein by reference.

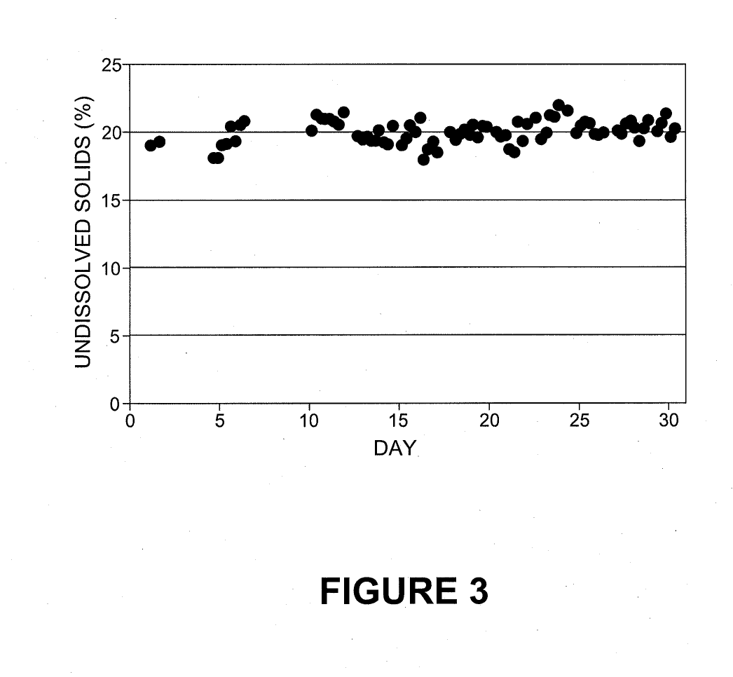

The undissolved dry solid content of the pretreated feedstock was measured over a one month period of operation. The results are shown in FIG. 3. The figure shows that, over the time period in which the measurements were taken, there were no large deviations in the solids concentration of the pretreated feedstock. This shows that the process can be operated at constant consistency over a prolonged time period.

A sample of the pretreated feedstock was also tested for its ability to be hydrolyzed by cellulase enzymes to produce glucose. By using the methods described herein to produce a pretreated feedstock, a high yield of glucose can be obtained.

In this example, the pretreated feedstock was hydrolyzed using cellulase enzymes secreted by Trichoderma reesei. The cellulase was produced by submerged liquid culture fermentation of Iogen Energy strain P1380H using methods described in US 2010/0304438, which is incorporated herein by reference. The filtered fermentation broth was de-salted using Biospin® columns (Bio-Rad) following the manufacturer's protocol. Total protein concentration of the desalted enzyme was assayed using a BCA kit (Sigma-Aldrich®) with a bovine serum albumin (Sigma-Aldrich®) control.

The cellulose of the pretreated wheat straw was hydrolyzed in a batch reaction using the cellulolytic enzyme systems obtained as described above. Pretreated wheat straw was hydrolyzed with 30 mg of cellulase per gram of cellulose in reactions at 50° C. and pH 5.0, with 250 rpm orbital shaking, in a total reaction volume of 50 mL. After 165 h, an aliquot was removed from the reaction; the reaction was well mixed during sampling to ensure homogeneity of solids and liquid the sample. The reaction was stopped in the aliquot sample by incubating it in a 100° C. hot block for 5 minutes.

The liquid fraction of the inactivated sample was analyzed for glucose concentration to determine the extent of cellulose conversion. The glucose concentration was determined using a coupled enzymatic assay based on glucose oxidase and horseradish peroxidases using methods known in the art. (See Trinder, 1969, Ann. Clin. Biochem., 6:24-27, which is incorporated herein by reference). The amount of glucose-equivalents present in the cellulose at the start of the reaction was determined in a separate acid hydrolysis of the pretreated cellulose to glucose, using methods known to those skilled in the art. The conversion calculation included correction terms for the effect of glucose on the density of the solution and the volume-exclusion effect of non-hydrolyzable lignin present in the reaction.

The calculated conversion of the cellulose in the pretreated feedstock was 90%, indicating that the pretreatment was effective in producing a cellulosic substrate from which high glucose yields can be recovered.

1. A method for producing a pretreated or hydrolyzed lignocellulosic feedstock comprising:
(i) feeding a lignocellulosic feedstock to a plug formation device and forming a feedstock plug therein;
(ii) feeding the plug or segments thereof into an elongate chamber having at least a portion thereof that is cylindrical, said chamber having steam addition means for direct steam addition and a rotating shaft mounted therein having one or more disintegrating elements arranged thereon;
(iii) producing disintegrated feedstock particles in said elongate chamber by said disintegrating elements;
(iv) heating the disintegrated feedstock particles by contacting the particles with steam introduced through said steam addition means, wherein the operating pressure in the chamber is at least about 90 psia; and thereafter
(v) pretreating or hydrolyzing the disintegrated feedstock particles in a reactor to produce the pretreated or hydrolyzed lignocellulosic feedstock.

2. The method of claim 1, wherein the lignocellulosic feedstock is in the form of a slurry and is fed to a dewatering device, to produce a dewatered feedstock and, wherein, the dewatered feedstock is then fed to the plug formation device.

3. The method of claim 1, wherein the feedstock is pressurized and then fed to a combined dewatering and plug formation device and wherein the pressure of the feedstock at the inlet of the device is greater than about 45 psia.

4. The method of claim 3, wherein the disintegrating elements are arranged on said shaft so as to sweep the inner surface of at least a region of the chamber.

5. The method of claim 4, wherein the disintegrating elements continuously axially sweep the inner surface of at least a region of the chamber.

6. The method of claim 5, wherein the steam addition means comprises inlets for direct steam injection disposed along the length of the chamber.

7. The method of claim 6, wherein the chamber does not contain an indirect heating jacket.

8. The method of claim 7, wherein pretreating or hydrolyzing comprises the addition of chemical to the disintegrated feedstock particles.

9. The method of claim 8, wherein the chemical is acid or alkali.

10. The method of claim 9, wherein the distance between the inner surface of the chamber and the outer edge of the disintegrating element that is closest to the inner surface is less than about 10 percent of the inside diameter of the chamber.

11. The method of claim 10, wherein the speed of the outer edge of the disintegrating element that is closest to the inner surface of the chamber is about 200 m/min to about 1000 m/min.

12. The method of claim 11, wherein the speed is about 450 m/min to about 800 m/min.

13. A method for producing a pretreated or hydrolyzed lignocellulosic feedstock comprising:
(i) feeding a lignocellulosic feedstock to a plug formation device and forming a feedstock plug therein;
(ii) feeding the plug or segments thereof from the plug formation device into an elongate chamber having at least a portion thereof that is cylindrical, said chamber having steam addition means for direct steam addition and a rotating shaft mounted therein having one or more disintegrating elements arranged thereon, wherein the disintegrating elements are arranged on said shaft so as to sweep the inner surface of at least a region of the chamber;
(iii) producing disintegrated feedstock particles in said elongate chamber by said disintegrating elements;
(iv) heating the disintegrated feedstock by contacting the particles with steam introduced through said steam addition means, wherein the operating pressure in the chamber is at least about 90 psia; and thereafter
(v) pretreating or hydrolyzing the disintegrated feedstock particles in a pretreatment reactor to produce the pretreated or hydrolyzed lignocellulosic feedstock.

14. A lignocellulosic feedstock composition comprising:
(i) disintegrated lignocellulosic feedstock particles;
(ii) about 15 to about 35 wt % undissolved solids, wherein the undissolved solids comprise between about 20 and about 60 wt % cellulose and between about 10 and about 30 wt % xylan; and
(iii) a mineral or organic acid;
wherein said feedstock particles are not primarily derived from wood chips or pulp, and wherein the pH of the feedstock composition is between about 0.5 and about 4.5.

15. The lignocellulosic feedstock composition of claim 14, wherein the temperature of the composition is between about 100° C. and about 280° C.

16. The lignocellulosic feedstock composition of claim 15, wherein the lignocellulosic feedstock is bagasse or sugar cane straw.

17. The lignocellulosic feedstock composition of claim 16, wherein the lignocellulosic feedstock composition comprises about 15 wt % to about 30 wt % undissolved solids.

18. A method comprising pretreating the lignocellulosic feedstock composition of claim 15.

19. A pretreated lignocellulosic feedstock composition produced according to the method of claim 18, wherein at least 70% of the cellulose in the pretreated lignocellulosic feedstock is converted to glucose, as measured when hydrolyzed with Trichoderma reesei cellulase enzymes, and wherein the pretreated lignocellulosic feedstock is produced from sugar cane bagasse or sugar cane straw.

20. A method for producing an alcohol from a lignocellulosic feedstock comprising:
(i) feeding a lignocellulosic feedstock in the form of a slurry to a plug formation device and forming a feedstock plug therein, wherein the plug or segments thereof exiting the plug formation device have an undissolved dry solids content between about 20 wt % and about 35 wt %;
(ii) pretreating the lignocellulosic feedstock after step (i) to produce a pretreated lignocellulosic feedstock having an undissolved dry solids content of between about 15 and 25 wt %;
(iii) enzymatically hydrolyzing the pretreated lignocellulosic feedstock to produce a solution comprising at least glucose; and
(iv) fermenting at least the glucose to produce the alcohol,
wherein the lignocellulosic feedstock is selected from cultivated crops, sugar processing residues and agricultural residues having an ash content of greater than 0.5% (w/w).

21. The method of claim 20, wherein the slurry fed to the plug formation device is a soaked feedstock slurry.

22. The method of claim 21, wherein the lignocellulosic feedstock is sugar cane bagasse or sugar cane straw.

23. The method of claim 20, 21 or 22, claim 21, wherein the lignocellulosic feedstock has an ash content of between 1.5% and 15% (w/w).

24. A method for producing a pretreated or hydrolyzed lignocellulosic feedstock comprising:
(i) soaking a lignocellulosic feedstock with an aqueous solution to produce a soaked lignocellulosic feedstock, wherein said lignocellulosic feedstock does not primarily contain wood chips or pulp;
(ii) feeding the soaked lignocellulosic feedstock to a plug formation device and forming a feedstock plug therein, wherein the plug or segments thereof exiting the plug formation device have an undissolved dry solids content between about 20 wt % and about 35 wt %;
(iii) disintegrating the plug or segments thereof to produce disintegrated feedstock particles and heating the disintegrated feedstock particles; and thereafter
(iv) pretreating or hydrolyzing the disintegrated feedstock particles in a reactor to produce the pretreated or hydrolyzed lignocellulosic feedstock.

25. The method of claim 24, wherein the soaked feedstock is partially dewatered prior to being fed to the plug formation device.

26. The method of claim 24, wherein the soaked feedstock is partially dewatered in a pressurized screw press prior to plug formation.

27. The method of claim 23, wherein the lignocellulosic feedstock is not leached or washed prior to step (i) in order to remove greater than 50 wt % of the ash.
